(19) United States
(12) Patent Application Publication
(10) Pub. No.: US 2022/0040427 A1
(43) Pub. Date: Feb. 10, 2022

(54) DEVICE AND PROCESS FOR MEASURING THE LUNG COMPLIANCE

(71) Applicant: Drägerwerk AG & Co. KGaA, Lübeck (DE)

(72) Inventors: Eckhard TESCHNER, Lübeck (DE); Frank RALFS, Lübeck (DE)

(21) Appl. No.: 17/394,922

(22) Filed: Aug. 5, 2021

(30) Foreign Application Priority Data
Aug. 7, 2020 (DE) 10 2020 120 900.2

(51) Int. Cl. A61M 16/00

(52) U.S. Cl. CPC A61M 16/024; A61M 2230/46; A61M 2230/65; A61M 2205/3344

(57) ABSTRACT

A device and a process determine a value indicative of a respective regional compliance of lungs of a patient (P) in a plurality of different regions of the lungs. An airway pressure sensor (3) measures a value indicative of the pressure (Paw), which is variable over time, at the airway of the patient (P). A difference between the end-inspiratory transpulmonary pressure and the end-expiratory transpulmonary pressure is determined. An EIT measuring device (17) measures by electrical impedance tomography (EIT) a change in volume of a lung region. The difference between the end-inspiratory volume and the end-expiratory volume of the lung region is determined with the use of signals of the EIT measuring device (17). A quotient of the volume difference for the region in question and the pressure difference present at the lungs is calculated as the value indicative of the regional compliance of the lung region.

### CROSS REFERENCE TO RELATED APPLICATIONS

This application claims the benefit of priority under 35 U.S.C. §119 of German Application 10 2020 120 900.2, filed Aug. 7, 2020, the entire contents of which are incorporated herein by reference.

### TECHNICAL FIELD

The present invention pertains to a device and to a process for determining a value indicative of the respective regional compliance of the lung of a patient in a plurality of different regions of the lungs.

### TECHNICAL BACKGROUND

The term “respiratory system” of the patient will be used below. The respiratory system comprises the lungs and the chest wall of the patient.

When a patient is breathing spontaneously and/or is ventilated mechanically, the following two undesired situations, each of which may damage the lungs of the patient, shall be avoided:
The pneumatic pressure exerted on the lungs is too low, and the lungs therefore collapse.
The pressure exerted is too high, and the lungs are hyperdistended.

An approach to controlling a ventilator as a function of the compliance of the lungs is described in Zhanqi Zhao et al.: “Positive End-Expiratory Pressure Titration with Electrical Impedance Tomography and Pressure-Volume Curve in Severe Acute Respiratory Distress Syndrome,” Annals of Intensive Care, 9, No. 1 (December 2019), available at https://doi.org/10.1186/s13613-019-0484-0, downloaded on Jun. 12, 2020. Other approaches to determining mechanical/pneumatic properties of the lungs of a patient and/or to controlling a ventilator are described in
Ola Stenqvist, Per Persson, and Stefan Lundin: “Can We Estimate Transpulmonary Pressure without an Esophageal Balloon?—Yes,” Annals of

Translational Medicine, 6, No. 19 (October 2018): 392 ff, available at https://doi.org/10.21037/atm.2018.06.05, downloaded on Jun. 12, 2020.
Giacomo Bellani et al.: “Plateau and Driving Pressure in the Presence of Spontaneous Breathing,” Intensive Care Medicine, 45, No. 1 (January 2019): 97-98, available at https://doi.org/10.1007/s00134-018-5311-9, downloaded on Jun. 12, 2020,
Daniel Talmor et al.: “Mechanical Ventilation Guided by Esophageal Pressure in Acute Lung Injury,” New England Journal of Medicine, 359, No. 20 (Nov. 13, 2008): 2095-2104, available at https://doi.org/10.1056/NEJMoa0708638, downloaded on Jun. 12, 2020,
Jeremy R. Beitler et al.: “Effect of Titrating Positive End-Expiratory Pressure (PEEP) With an Esophageal Pressure-Guided Strategy vs. an Empirical High PEEP-FiO₂ Strategy on Death and Days Free From Mechanical Ventilation Among Patients With Acute Respiratory Distress Syndrome: A Randomized Clinical Trial,” JAMA, Feb. 18, 2019, available at https://doi.org/10.1001/jama.2019.0555, downloaded on Jun. 12, 2020.

The compliance of the lungs may differ from one region of the lungs to the next. It is known, for example, from DE 10 2005 031 752 B4 (corresponding to U.S. Pat No. 7,941,210 B2) that the lung can be measured by means of impedance tomography, preferably by means of a so-called EIT belt, which is placed around the body of the patient.

It is proposed in Eduardo L. V. Costa et al.: “Bedside Estimation of Recruitable Alveolar Collapse and Hyperdistention by Electrical Impedance Tomography,” Intensive Care Medicine, 35, No. 6 (June 2009), 1132-1137, available at https://doi.org/10.1007/s00134-009-1447-y, downloaded on Jun. 12, 2020, that the compliance or elasticity (compliance) of the respiratory system of a patient be analyzed while a variable end-expiratory pressure (PEEP) is being set. Different values are set on a trial basis and the extent of the reduction of the compliance of the respiratory system is investigated in different regions, and this reduction is interpreted as a hyperdistention or as a collapse of the lungs.

### SUMMARY

A basic object of the present invention is to provide a device and a process which are capable of measuring the compliance of the lungs better than prior-art devices and processes can.

The object is accomplished by a device having the features of the device according to the invention and by a process having the features of process according to the invention. Advantageous embodiments are described. Advantageous embodiments of the device are, insofar as meaningful, also advantageous embodiments of the process according to the present invention and vice versa.

The present invention pertains to a device and to a process, which are capable of automatically determining a value indicative of (a parameter of) the respective regional compliance of the lungs of a patient in at least two different regions of the lungs. The regional compliance is the compliance of the lungs in a region of the lungs, wherein each lung region is predefined and comprises a respective part of the lung each but not the entire lung. Preferably the lung regions are disjoint, i.e. they do not overlap. Each region of the lungs, whose regional compliance shall be determined, is specified such that the regional compliance in the entire lung region is considered to be equal everywhere at an accuracy sufficient for the application. The determined regional compliance of a lung region may vary over time, for example, because the state of the patient changes or based on different settings of a ventilator, which mechanically ventilates the patient. The respective regional compliance may vary from one lung region to the next at a given time.

The device according to the present invention comprises an EIT measuring device. This EIT measuring device is capable of measuring a value indicative of (a parameter of) the change in the volume of a lung region and to use an electrical impedance tomography (EIT) process for this. Thanks to the EIT measuring device, a value indicative of a change in the volume of each lung region can be measured for the respective change in the volume of each lung region.

The device according to the present invention comprises, furthermore, an airway pressure sensor. This airway pressure sensor is capable of measuring a value indicative of (a parameter) of the pressure, which is variable over time, at or in the airway of the patient, preferably a pneumatic parameter. This pressure results from the intrinsic breathing activity of the patient and/or from a mechanical ventilation of the patient, which is carried out by a ventilator. The intrinsic breathing activity of the patient may result from a spontaneous breathing of the patient as well as from an intrinsic breathing activity stimulated from the outside.

A data processing control device of the device according to the present invention is capable of receiving and processing signals from the EIT measuring device and from the airway pressure sensor and it calculates a respective parameter (a respective value indicative of) each for the regional compliance of the lungs in this area. The control device preferably calculates the respective value indicative of the regional compliance for four lung regions, which are arranged in the manner of four quadrants, or for eight lung regions. The control device calculates a quotient
of a volume difference for this lung region and
of a pressure difference, which is present at the lungs of the patient as this value indicative of the regional compliance of a lung region.

The control device calculates as the volume difference a value indicative of (a parameter) of the difference between the end-inspiratory volume and the end-expiratory volume of this region of the lungs. The end-inspiratory volume occurs at the end of an inhalation process of the patient, and the end-expiratory volume at the end of an exhalation process. The lungs are known to expand during inhalation and to contract again during exhalation, so that the end-inspiratory volume is larger than the end-expiratory volume. In order to determine these two volumes, the control device uses signals of the EIT measuring device. The volume difference may, of course, differ from one lung region to the next and it may also vary over time.

The control device determines as a pressure difference a value indicative of (a parameter of) the difference between the end-inspiratory transpulmonary pressure and the end-expiratory transpulmonary pressure of the entire respiratory system of the patient. The term “transpulmonary pressure” designates a pressure that acts on the lungs from the inside or also from the outside. This transpulmonary pressure is the difference between
the pressure that acts on the entire respiratory system of the patient and
the pressure acting on the pleural cavity of the patient.

This pleural cavity encloses the lungs of the patient. The respiratory system comprises the lungs and the chest wall of the patient, and the pleural cavity is located between the lungs and the chest wall.

The control device uses signals of the airway pressure sensor to determine the pressure difference. These signals are a parameter of the entire pressure that acts on the respiratory system and is generated
only by the intrinsic breathing activity, i.e., by the spontaneous breathing or by an externally stimulated, intrinsic breathing activity of the patient,
only by the mechanical ventilation of the patient by a ventilator or
by a superimposition of the intrinsic breathing activity and the mechanical ventilation.

Knowledge of the extent of compliance of different regions of the lungs of a patient can be used for the proper setting of a ventilator for the mechanical ventilation of this patient. The ventilator performs a series of ventilation strokes as a function of at least one operating parameter. The operating parameters that can be set on the ventilator include, for example,
the positive end-expiratory pressure (PEEP),
the end-inspiratory pressure,
a desired lung volume (tidal volume), i.e., the volume of the gas that is fed to the patient during a ventilation stroke of the ventilator, or
a desired volume flow between the ventilator and the patient.

The ventilator shall be set especially such that a hyperdistention of the lungs is avoided during a mechanical ventilation, on the one hand, and that collapse of the lungs is, on the other hand, ruled out. Knowledge of the possible extent of compliance of the lungs of a patient can also be used for a patient monitor and/or for the automatic monitoring of the patient. The patient monitor receives and uses measured values from the device according to the present invention and it preferably displays the received measured values in a form perceptible by a person, especially visually on a display unit.

The compliance of a pneumatic system with variable volume is known to result from the quotient
of a pressure difference acting on the system (denominator of the quotient) and
of the change in the volume (numerator of the quotient), which change results from this pressure difference.

A pressure difference and a brought-about volume change are determined according to the present invention.

If a patient is supplied with the needed breathing air exclusively by mechanical ventilation, i.e., if the patient is fully anesthetized, the current signal value of the sensor for the airway pressure provides a good value indicative of the pressure and hence for the load that currently acts on the lungs. However, a patient is frequently supplied with breathing air exclusively by mechanical ventilation only for as short a time as possible. As long as possible, the patient also performs an intrinsic breathing activity, namely, a spontaneous breathing and/or a stimulated breathing, i.e., a breathing with his own respiratory muscles, in addition to the mechanical ventilation or even instead of a mechanical ventilation. The mechanical ventilation consequently supports the intrinsic breathing activity. The current signal value of the airway pressure sensor alone is not sufficient in this case to reliably determine the pressure currently acting on the lungs and hence also the acting pressure difference. The end-inspiratory transpulmonary pressure and the end-expiratory transpulmonary pressure are rather determined according to the present invention, and the difference between these two transpulmonary pressures is used as the pressure difference.

The brought-about volume change of the entire lung can be determined by means of a volume sensor or a volume flow sensor. For example, the current volume flow into or out of the lungs of the patient is measured several times one after another, and integrated over the measured values.

However, the compliance of the lungs may differ substantially from one lung region to the next. As a consequence, in many cases a global change in the volume of the lungs does not suffice to determine a regional compliance. The respective compliance of at least two different lung regions and preferably of four lung regions, which are arranged like four quadrants, or even of eight different lung regions, is therefore determined according to the present invention. Signals of the EIT measuring device are used for this according to the present invention. These signals yield a respective value indicative of the regional volume change of the lung region based on the breathing/ventilation for different lung regions. A respective brought-about volume change each is preferably determined for each lung region or at least for each region of a relevant part of the lungs.

The device according to the present invention and the process according to the present invention may, of course, also be used during the time period in which the patient is supplied with breathing air exclusively by mechanical ventilation. It is possible but not necessary thanks to the present invention to switch over between two different modes, namely,
a mode for the exclusive mechanical ventilation and
a mode for a superimposition of mechanical ventilation and intrinsic breathing activity, i.e., the supportive mechanical ventilation, during the determination of the regional compliances.

The airway pressure sensor is preferably located outside the body of the patient. For example, a part of the sensor is located in front of the mouth of the patient and branches off air from a breathing air stream or from a ventilation circuit. An analysis unit of the sensor is located at a distance in space from the patient, for example, at or in a ventilator or a patient monitor. Thanks to this embodiment, it is not necessary to bring a part of the airway pressure sensor from time to time into the body of the patient and later to remove it. However, an airway pressure sensor with a reading recorder in the body of the patient may be used for the present invention as well.

The pressure in the pleural cavity cannot be measured directly in a person and in many other living beings. In one embodiment, the device additionally comprises a pneumatically operating sensor for the pressure in the esophagus. This sensor provides a value indicative of the pressure in the esophagus of the patient. The measured pressure in the esophagus is a good approximation for the pressure in the pleural cavity. As was already described, the difference between the airway pressure and the pressure in the pleural cavity, which cannot be measured directly, is a value indicative of the transpulmonary pressure. According to this embodiment, the difference between
the airway pressure, which is measured by the airway pressure sensor, and
the esophageal pressure, which is measured by the esophageal sensor, is used as the transpulmonary pressure. To determine the pressure difference, an end-inspiratory transpulmonary pressure as a difference between the end-inspiratory airway pressure and the end-inspiratory esophageal pressure is used at least once as well as an end-expiratory transpulmonary pressure as a difference between the end-expiratory airway pressure and the end-expiratory esophageal pressure is used at least once. The pressure difference is preferably determined anew at each scanning time of the two sensors.

In a preferred embodiment, this esophageal pressure sensor comprises a catheter and a measuring balloon, both of which are positioned in the esophagus of the patient. The measuring balloon is in a fluid connection with the catheter, and the catheter is in a fluid connection with an analysis unit located outside the patient.

In a preferred embodiment, the present invention is used while the patient is being ventilated mechanically by a ventilator (supporting or mandatory ventilation). The ventilator may be configured as an anesthesia apparatus and it may additionally anesthetize the patient by means of at least one anesthetic. The mechanical ventilation is carried out such that the end-expiratory pressure (PEEP), which shall be brought about by the ventilator, is set at a certain, predefined value. This end-expiratory pressure shall prevail in the respiratory system of the patient at the end of an exhalation process. The airway pressure sensor is capable of measuring the actual end-expiratory pressure, so that a regulation of the mechanical ventilation with the aim of making the actual value for the end-expiratory pressure equal to the predefined value is possible, even as an automatic regulation. At least two, preferably more than two different values are preferably predefined for the end-expiratory pressure to be brought about, and the ventilator is regulated to this respective value.

In a variant of this embodiment, the control device automatically predefines a first required value and at least one second required value for the end-expiratory pressure. The mechanical ventilation is carried out with the regulation goal to ensure that the actual value for the end-expiratory pressure be at first equal to the first predefined value and then to be equal to the second predefined value or to a second predefined value, wherein these two values are different from one another. It is possible to predefine more than two values for the end-expiratory pressure and to set the mechanical ventilation one after another to each of these at least three values.

In one application, a fluid connection is used between the ventilators and the patient in order to automatically determine a desired operating value for the end-expiratory pressure, which shall be brought about by the ventilator during the mechanical ventilation. The regional compliance of a region of the lungs, which is determined according to the present invention, depends on the value set for the end-expiratory pressure. The respective regional compliance of the lungs is determined according to the present invention for each of the at least two values of the end-expiratory pressure and for a plurality of regions. The respective overall lung compliance effected by the different values for the end-expiratory pressure is determined by cumulating over regional compliances. Cumulation is performed in a suitable manner over these regional compliances.

The value of the expiratory pressure that leads to the highest regional compliance of the lung region in question is determined for a plurality of regions of the lungs in one embodiment. This optimal valve varies, as a rule, from one lung region to the next. A relative compliance or stiffness or elastance of the lung region is calculated for each lung region taken into consideration and for a plurality of values of the end-expiratory pressure. The optimal value for a lung region is set automatically as a function of the determined relative compliances. This relative value equals 100% for the optimal value that leads to the maximum regional compliance. Cumulation is then performed over the lung regions whose regional compliance is determined.

The embodiment just described, in which the mechanical ventilation is set one after another at different values for the end-expiratory pressure, may be combined with the above-described embodiment, in which the transpulmonary pressure is determined as the difference between the airway pressure and the esophageal pressure.

In a different embodiment, the necessity to equip the patient with a pneumatic sensor for the pressure in the esophagus is avoided. This alternative embodiment can likewise be used when the patient is ventilated mechanically or when the end-expiratory pressure reached is set one after another at at least two different values. Each value for the end-expiratory pressure leads to a respective resulting end-expiratory lung volume, i.e., to the lung volume present at the end of an exhalation process. The difference between the two end-expiratory lung volumes is calculated. In addition, the difference between the two values for the end-expiratory pressure is calculated. The quotient of the volume difference and the pressure difference provides a value indicative of the difference between the end-inspiratory transpulmonary pressure and the end-expiratory transpulmonary pressure.

The airway pressure sensor and the control device may be parts of a ventilator or of an anesthesia apparatus. A fluid connection, optionally a ventilation circuit, is established at least from time to time between the patient and the ventilator or the anesthesia apparatus. The EIT measuring device is in a data connection with the ventilator or with the anesthesia apparatus.

The present invention pertains, furthermore, to a system which is capable of ventilating a patient mechanically. The system comprises a ventilator, which may be configured as an anesthesia apparatus, as well as a device according to the present invention. The ventilator is capable of carrying out a mechanical ventilation of the patient and it preferably carries out a series of ventilation strokes during the mechanical ventilation. The ventilator uses for this mechanical ventilation the regional compliances of the lungs of the patient, which are determined according to the present invention. Preferred embodiments of the device according to the invention are also preferred embodiments of the ventilating system. The ventilator preferably generates a respective value each for at least one operating parameter of the ventilator as a function of the regional compliances. The operating parameter is, for example,
the tidal volume, i.e., the volume of the breathing air delivered to the body of the patient during a ventilation stroke,
the maximum volume flow per minute,
the maximum ventilation pressure during a ventilation stroke,
the pressure at the end of an inhalation process and before the subsequent exhalation process, often called plateau pressure,
the end-expiratory pressure (PEEP), i.e., the pressure that the lungs have at the end of a ventilation stroke,
the ramp time, i.e., the time that elapses during a ventilation stroke until the ventilation pressure reaches the maximum value,
the duration of an inhalation phase, i.e., a phase during which breathing air flows to the patient,
the duration of an exhalation phase, i.e., the duration of a phase during which breathing air flows away from the patient, or
the frequency at which ventilation strokes are carried out.

A predefined calculation rule is preferably used to calculate the value of the operating parameter as a function of the regional compliances. The calculation rule is predefined such that neither is a region of the lungs hyperdistended, nor does it collapse into itself based on an excessively low pressure. In one embodiment, the ventilator outputs the calculated value, and a user can confirm the value or overwrite it with another value. In another embodiment, the ventilator uses the automatically calculated value.

The present invention will be described below on the basis of exemplary embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a view showing a patient and a ventilator, which mechanically ventilates the patient;

FIG. 2 is a schematic view of sensors on a display unit of the ventilator;

FIG. 3 is a display view showing the time course (time curve) of a plurality of vital parameters of the patient;

FIG. 4 is a schematic view showing an output of three values of the transpulmonary pressure;

FIG. 5 is a schematic view showing an exemplary EIT measuring device; and

FIG. 6 is a view showing a maneuver in which the end-expiratory pressure is reduced step by step, as well as the respective resulting regional compliance of the lungs.

### DESCRIPTION OF PREFERRED EMBODIMENTS

Referring to the drawings, the present invention is embodied in the exemplary embodiment by means of a device, which comprises
a ventilator,
an EIT measuring device,
additional sensors and
a higher-level data processing control device.
   The higher-level control device receives signals from the EIT measuring device and from the additional sensors and controls the ventilator as a function of these signals, especially an actuator of the ventilator, which carries out the ventilation strokes.

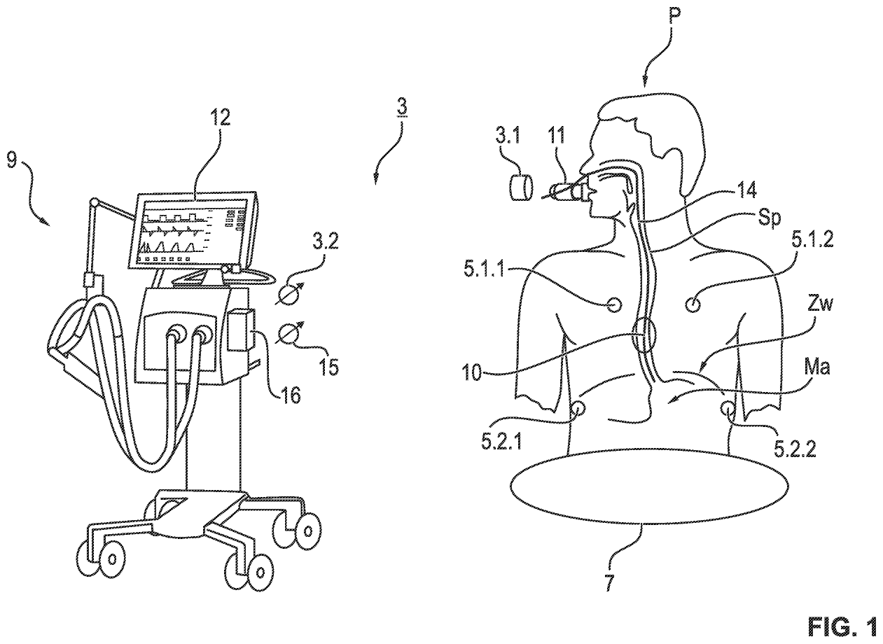

FIG. 1 schematically shows a patient P, who is being ventilated mechanically. The esophagus Sp, the stomach Ma and the diaphragm Zw of the patient P are shown. In addition,
a ventilator 9,
a flexible measuring catheter 14,
a connection piece 11 in the mouth of the patient P,
an EIT belt 7,
a plurality of exemplary sensors, and
a higher-level control device 16 comprising one or more processors and memory, which will be described below, are shown.

A fluid connection, not shown, connects the patient P to the ventilator 9. A gas or a gas mixture can flow through this fluid connection from the ventilator 9 to the patient P. A ventilation circuit is optionally established between the patient P and the ventilator 9, i.e., breathing air, which has been exhaled by the patient P, flows back to the ventilator 9.

Different sensors measure different vital parameters of the patient P. A pneumatic sensor 3 comprises a transducer 3.1 comprising an opening, which is arranged in the vicinity of the mouth of the patient P and branches off air from the fluid connection between the patient P and the ventilator 9. The branched-off air is transferred via a hose (tube) to a pressure sensor 3.2, which measures a value indicative of the airway pressure P_{aw} (pressure in airway). In one embodiment, the transducer 3.1 is arranged in or at a Y-piece close to the connection piece 11. A sensor 15 at the ventilator 9 optionally measures a parameter of the volume per unit of time of the flow Vol′ of breathing air from the ventilator 9 to the patient P or back from the patient P to the ventilator 9. By analyzing measured values of the sensor 3 and/or of the sensor 15, the ventilator 9 is capable of determining when an inhalation process (inhalation) of the patient P begins and when it ends and when an exhalation process (exhalation) begins and when it ends.

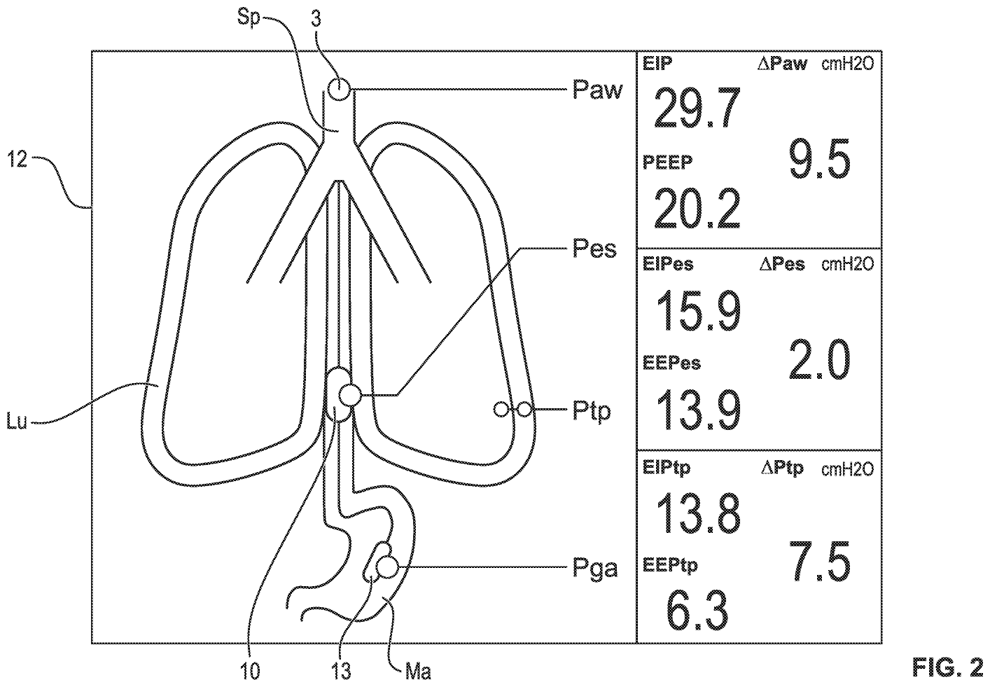

The measuring catheter 14 is placed into the esophagus Sp. The measuring catheter 14 begins in the connection piece 11. A probe 10 in the esophagus Sp of the patient P, preferably comprising a measuring balloon, measures a value indicative of the pneumatic pressure Pₑₛ (pressure in esophagus), which is variable over time, in the esophagus Sp. The probe 10 is in a fluid connection with the connection piece 11 via the measuring catheter 14 or is a part of the measuring catheter 14. In one embodiment, the probe 10 is positioned at the transition between the esophagus Sp and the stomach Ma and comprises a measuring balloon and optionally two measuring balloons. The measuring balloon of the probe 10 or a measuring balloon of the probe 10 is located in the lower region of the esophagus Sp and is deformed as a function of the pressure Pₑₛ in the esophagus Sp. The optional other measuring balloon is located behind the cardiac orifice in the stomach Ma and is deformed depending on the gastric pressure P_{ga} in the stomach Ma. It is also possible that an additional gastric probe 13 in the form of a measuring balloon is placed into the stomach Ma, cf. FIG. 2. A value indicative of the gastric pressure P_{ga} can be measured in the stomach Ma in both cases.

In addition, a plurality of measuring electrodes are preferably attached to the chest of the patient P. FIG. 1 shows as an example a pericardial pair 5.1.1, 5.1.2 of measuring electrodes as well as a pair 5.2.1, 5.2.2 of measuring electrodes near the diaphragm. In addition, a measuring electrode for ground, not shown, is attached to the chest of the patient P. An electrocardiogram (EKG) and/or an electromyogram (EMG) of the patient P is generated by means of these optional measuring electrodes 5.1.1, . . . , 5.2.2. The EKG and/or the EMG are optionally outputted during the mechanical ventilation in a form perceptible by a person. The measuring electrodes 5.1.1 through 5.2.2 are not necessarily needed for the process described below.

In addition, a belt 7 is placed around the body of the patient P for an electrical impedance tomography (EIT), which will be described below. This EIT belt 7 belongs to an EIT measuring device, which comprises in the exemplary embodiment an additional sensor for the airway pressure P_{aw} (not shown).

The patient P is ventilated mechanically by the ventilator 9 at least from time to time. A fluid connection, optionally a ventilation circuit, is established between the patient P and the ventilator 9. The ventilator 9 carries out ventilation strokes and thereby delivers breathing air through the fluid connection to the mouthpiece 3 and into the lungs of the patient P. This breathing air is optionally mixed with at least one anesthetic, so that the patient P is anesthetized at least partially.

A display unit 12, which is in a data connection with the ventilator 9, displays
a schematic view of the lungs Lu and of the esophagus Sp of the patient P,
schematically the respective position at which a vital parameter of the patient P is measured, and
current values of different vital parameters of the patient P and of additional parameters during the mechanical ventilation of the patient P.
   FIG. 2 illustrates as an example a view of the values of different viral parameters, which are variable over time, on the display unit 12. Here,
P_{aw} (airway pressure) is a value indicative of the airway pressure,
Pₑₛ (pressure in esophagus, esophageal pressure) is a value indicative of the pressure in the esophagus Sp,
P_{ga} (gastric pressure) is a value indicative of the gastric pressure in the stomach Ma,
Pₜₚ (transpulmonary pressure) is a value indicative of the transpulmonary pressure, which will be explained below,
EIP is the current value of the end-inspiratory pressure at the airway of the patient P, namely, of the pressure P_{aw} at the end of an inhalation process and before the next exhalation process, and this end-inspiratory pressure is also often called a plateau pressure Pplat,
PEEP (positive end-expiratory pressure), the current value of the end-expiratory pressure at the airway, namely, of the pressure Pₐ, at the end of an exhalation process and before the next inhalation process of the patient P,
ΔP_{aw}, the difference between the current values of the end-inspiratory pressure EIP and of the end-expiratory pressure PEEP at the airway, i.e., ΔP_{aw}=EIP−PEEP, wherein this difference is also called driving pressure,
EIPₑₛ (end-inspiratory esophageal pressure), the current value of the end-inspiratory pressure in the esophagus Sp, namely, of the pressure Pₑₛ at the end of an inhalation process and before the next exhalation process of the patient P,
EEPₑₛ (end-expiratory esophageal pressure), the current value of the end-expiratory pressure in the esophagus Sp, namely, of the pressure Pₑₛ at the end of an exhalation process and before the next inhalation process of the patient P,
ΔPₑₛ, the difference between the current values of the end-inspiratory pressure EIPₑₛ and of the end-expiratory pressure EEPₑₛ in the esophagus Sp, i.e., ΔPₑₛ=EIPₑₛ−EEPₑₛ,
EIPₜₚ (end-inspiratory transpulmonary pressure), the current value of the end-inspiratory transpulmonary pressure, namely, of the transpulmonary pressure Pₜₚ at the end of an inhalation process and before the next exhalation process of the patient P,
EEPₜₚ (end-expiratory transpulmonary pressure), the current value of the end-expiratory transpulmonary pressure, i.e., the pressure Pₜₚ at the end of an exhalation process and before the next inhalation process of the patient P,
ΔPₜₚ (transpulmonary driving pressure), the difference between the current values of the end-inspiratory transpulmonary pressure EIPₜₚ and the end-expiratory transpulmonary pressure EEPₜₚ, i.e., ΔPₜₚ=EIPₜₚ−EEPₜₚ,
EEP_{ga} (end-expiratory gastric pressure, not shown), the current value of the end-expiratory gastric pressure, i.e., the gastric pressure P_{ga} at the end of an exhalation process and before the next inhalation process of the patient P, and
P_{di} (transdiaphragmatic pressure, likewise not shown), the current value of the pressure at the diaphragm Zw as a difference between the current values of the gastric pressure P_{ga} and the end-inspiratory esophageal pressure EEPₑₛ in the esophagus Sp at the end of an exhalation process, i.e., P_{di}=P_{ga}−EEPₑₛ.

The airway pressure P_{aw} is measured by the pneumatic sensor 3, which is arranged in one embodiment in the Y-piece in front of the connection piece 11. The probe 10 in the esophagus Sp measures the pressure Pₑₛ in the esophagus Sp. For example, the deformation of the balloon, which belongs to the probe 10, is measured, and it acts as a value indicative of the pressure Pₑₛ in the esophagus Sp. Another measuring balloon of the probe 10 or an additional gastric probe 13 makes it possible to measure a value indicative of the pressure P_{ga} in the stomach Ma. For example, the deformation of one measuring balloon, which belongs to the probe 10, is measured and is used as a value indicative of the pressure Pₑₛ in the esophagus Sp. The deformation of the other measuring balloon of the probe 10 or the deformation of the separate gastric probe 13 yields a value indicative of the gastric pressure P_{ga}. The other signals are deduced from measured values of these sensors, which will be described farther below.

FIG. 2 shows, furthermore, a schematic view of the lungs Lu, of the stomach Ma and of the esophagus Sp of the patient P as well as the positions of the pneumatic sensor 3 and of the probes 10 and 13.

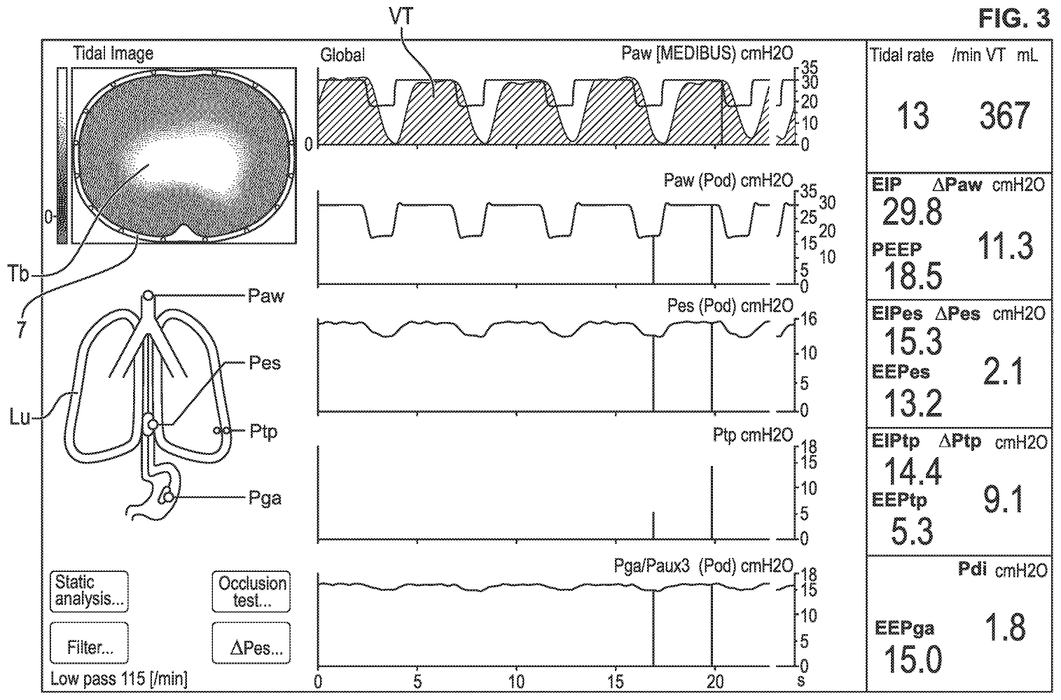

FIG. 3 shows as an example a view with a time course (time curves) for the following vital parameters:
In the left upper part, the current tidal image Tb, i.e., the current distribution of breathing air in the lungs Lu of the patient P, specifically in a horizontal cross-sectional plane, in which the EIT belt 7 extends, as well as an illustration of the EIT belt 7 around the chest or the stomach Ma of the patient P,
the global variation of an electrical resistance (impedance), which was measured by means of the EIT belt 7, and which is correlated with the tidal volume (tidal rate VT), as well as the time course of the airway pressure P_{aw}, measured by the pneumatic sensor of the EIT measuring device, in a view in the topmost row,
the airway pressure P_{aw}, measured by means of the pneumatic sensor 3 (second row from the top),
the pressure Pₑₛ in the esophagus Sp,
the transpulmonary pressure Pₜₚ, calculated preferably as a difference P_{aw}-Pₑₛ, and

the gastric pressure P_{ga} in the stomach Ma.

In addition, the measurement positions (left bottom) as well as current values of the vital parameters (right-hand column) are shown. The view in FIG. 3 is outputted, for example, on the display unit 12.

Different operating parameters, for example,
the end-inspiratory pressure EIP brought about by a ventilation stroke on the airway,
a set point for the end-expiratory pressure PEEP,
a set point for the plateau pressure,
a lower and/or upper limit for the airway pressure P_{aw},
the tidal volume VT achieved by a ventilation stroke, i.e., the brought-about increase in the lung volume, and
the start, duration, amplitude and/or frequency of ventilation strokes, for example,
   as a function of the intrinsic breathing activity of the patient P, can be set during the mechanical ventilation of the patient P on the ventilator 9.

The ventilator 9 preferably carries out a regulation, wherein a volume or a pressure is predefined in the fluid connection and it acts as a command variable of the control circuit (here of the ventilation circuit). The command variable may be variable over time.

When a patient P is breathing spontaneously and/or is ventilated mechanically, the lungs of the patient P expand and contract again. Each ventilation stroke of the ventilator 9 feeds air to the lungs and brings about an expansion of the lungs. The exhalation brings about a flow of air out of the lungs. This expansion and contraction of the lungs can be compared in a simplified manner to the process of pumping up the bicycle tube and then letting air again out of the tube. In case of considerably damaged or diseased lungs, the exhalation may lead to a collapse of the lungs into themselves.

Two situations critical for the patient P must be avoided, because each these situations may damage his lungs:
The lungs collapse into themselves (they collapse) because of an excessively low pressure and
the lungs are hyperdistended because of an excessively high pressure.

Both situations may occur simultaneously especially in case of damaged lungs, doing so in different regions of the lungs, which are damaged to different extents and/or in different manners.

The lungs and the chest wall of the patient P form together an expandable respiratory system.

If the patient P is fully anesthetized and is not carrying out any spontaneous breathing, the pressure that acts on the respiratory system of the patient P is exerted exclusively by the ventilator 9. The pressure P_{aw}, which is measured by the sensor 10 in the vicinity of the mouth of the patient P, is a good value indicative of the pressure that acts on the respiratory system in case of full anesthesia (the patient is not performing any intrinsic breathing activity at all). However, the situation in which the patient P is not fully anesthetized but breathes spontaneously at least from time to time during the mechanical ventilation or in which his intrinsic breathing activity is stimulated and the mechanical ventilation is superimposed to the intrinsic breathing activity (spontaneous breathing and/or stimulated intrinsic breathing activity) of the patient P should be taken into consideration. The intrinsic breathing activity is not taken sufficiently into account in many cases solely by the measured pressure P_{aw}.

The ventilation strokes of the ventilator 9 as well as the intrinsic breathing activity contribute to the expansion of the respiratory system. The entire pressure generated by the ventilator 9 brings about both a compliance of the lungs and an expansion of the chest wall. The jacketing of the lungs, called the pulmonary pleura, slides along the inner side of the chest wall during the breathing. The thin pleural cavity is located between the lungs and the chest wall. This pleural cavity is filled with a liquid and holds the two surfaces of the lungs and of the chest wall, which adjoin one another, together by capillary forces.

The difference between the total pressure, which is present at the respiratory system, and the pressure in the pleural cavity is a good value indicative of the pneumatic pressure that acts on the lungs during a mechanical ventilation, and it is thus a value indicative of the transpulmonary pressure. The pressure at the airway, preferably the airway pressure P_{aw} measured by the sensor 3, is used as the value indicative of the total pressure that is present. The pressure at the pleural cavity occurs between the outer side of the lungs (pulmonary pleura) and the inner side (costal pleura) of the chest wall. This difference takes into account quantitatively both the mechanical ventilation by the ventilator 9 and the intrinsic breathing activity of the patient P. Due to this difference being taken into consideration, the pressure acting on the lungs is separated by calculation from the pressure acting on the chest wall in the total pressure that acts on the respiratory system and the pressure acting on the lungs is determined as a result in an isolated manner.

The pleural cavity is a closed system. It is therefore impossible to measure this pleural pressure directly. A good approximation to the pleural pressure is the pressure Pₑₛ, which is measured by the probe 10 in the esophagus Sp of the patient P, providing that the probe 10 is positioned correctly in the esophagus Sp.

In order to prevent the lungs of the patient P from collapsing into themselves and/or from becoming hyperdistended, a value indicative of the mechanical compliance or elasticity of the lungs is measured. This elasticity is the quotient of the brought-about change ΔVol (increase or decrease) of the lung volume Vol and the change ΔP of the pressure present at the lungs, which is caused by this volume change, i.e., compliance=ΔVol/ΔP.

A change in the transpulmonary pressure Pₜₚ is used in the exemplary embodiment as the pressure difference that acts on the lungs and brings about a change in volume. Consequently, the quotient ΔVol/ΔPₜₚ is used as the value indicative of the elasticity of the lungs.

The transpulmonary pressure Pₜₚ is a value indicative of the mechanical stress to which the lungs are exposed based on the expansion and contraction occurring during breathing. The transpulmonary pressure Pₜₚ is preferably related to a reference value, for example, to the ambient air pressure, and may therefore assume negative values. The end-inspiratory transpulmonary pressure EIPₜₚ is a value indicative of the maximum expansion of the lungs, which occurs during breathing and during mechanical ventilation, and which must not become too great. The end-expiratory transpulmonary pressure EEPₜₚ is a value indicative of the minimal expansion and can indicate the risk that the lungs will collapse, especially at negative values of the expansion with respect to the environment.

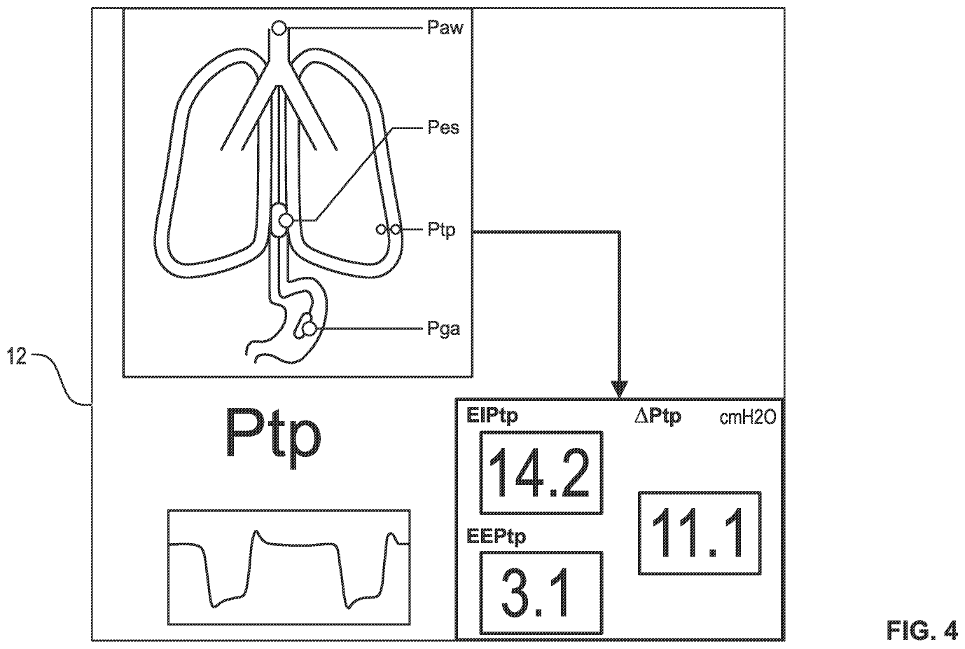

In one embodiment, the current values of these three parameters are displayed on the display unit 12. FIG. 4 shows as an example how the current values of the three parameters Pₜₚ, EIPₜₚ and EEPₜₚ as well as the value ΔPₜₚ are displayed on the display unit 12. In addition, FIG. 4 shows the time course of the transpulmonary pressure Pₜₚ.

The transpulmonary pressure Pₜₚ cannot be measured directly. In one embodiment, the transpulmonary pressure Pₜₚ is calculated as the difference between the pressure P_{aw} at the airway and the pressure Pₑₛ in the esophagus Sp, i.e.,

Pₜₚ=P_{aw}−Pₑₛ.

Correspondingly,

EIPₜₚ=EIP−EIPₑₛ and

EEPₜₚ=PEEP−EEPₑₛ

apply to the parameters shown in FIG. 2 through FIG. 4.

An alternative to determining the transpulmonary pressure Pₜₚ does not need the pressure in the esophagus Sp and therefore it makes do without a probe 10. Such a process is described in EP 2 397 074 B1 (corresponding U.S. Pat. No. 8,701,663 B2 and U.S. Pat. No. 9,655,544 B2 are incorporated herein by reference). The difference ΔPₜₚ between the end-inspiratory transpulmonary pressure EIPₜₚ and the end-expiratory transpulmonary pressure EEPₜₚ is determined in this alternative. The ventilator 9 carries out the mechanical ventilation such that two different values peep1 and peep2 are generated for the end-expiratory pressure PEEP one after another, wherein peep2 is the higher value. Each value leads to a respective value eelv1, eelv2 for the end-expiratory lung volume EELV. The pressure change peep2−peep1 brings about a change in volume, eelv2−eelv1. The difference ΔPₜₚ being sought is calculated, for example, according to the calculation rule

ΔPₜₚ=(eelv2−eelv1)/(peep2−peep1).

The compliance of the respiratory system comprising the lungs and the chest wall will hereinafter be designated by Cᵣₑₛₚ, the compliance of the lungs by C_{Lung} and the compliance of the chest wall by C_{ew}. The reciprocal value 1/C is also called elastance E. Here, 1/Cᵣₑₛₚ=1/C_{Lung}+1/C_{ew} as well as Eᵣₑₛₚ=E_{Lung}+EC_{ew}.

The compliance of a system is known to be the quotient of
the brought-about volume change and
the pressure, which brings about this volume change.

The respiratory system of the patient P is expanded by the pressure present during inhalation and it contracts again during exhalation. The tidal volume VT can be used as the volume difference AVol. The average compliance of the respiratory system can be described approximately by means of the following lung mechanical model equations:

Cᵣₑₛₚ=VT/ΔP_{aw},

C_{Lung}=VT/ΔPₜₚ,

C_{ew}=VT/ΔPₑₛ.

In one embodiment, the sensor 3 located in front of the mouth of the patient P measures, in addition to the airway pressure P_{aw}, the volume rate of flow Vol′ into and out of the airway of the patient P, i.e., the quantity of gas moving per unit of time. In another embodiment, the optional flow sensor 15 at the ventilator 9 measures this volume rate of flow Vol′. A volume change and especially the tidal volume VT, i.e., the volume that is taken up by the lungs during an individual inhalation process, can be deduced from this volume rate of flow Vol′. The size of the lungs increases by this volume.

It is possible to deduce the compliance of the lungs by means of this volume rate of flow Vol′ and the transpulmonary pressure P. This yields an average compliance. However, the compliance of the lungs varies, as a rule, from one region of the lungs to the next. Individual regions of the lungs may have a markedly lower compliance than other regions. This global method will not therefore lead, as a rule, to satisfactory results. The EIT measuring device with the EIT belt 7 is used in the exemplary embodiment to measure the local compliance of the lungs, i.e., the compliance in certain regions.

The basic idea behind such a measuring device is the following: A series of at least four, for example, 16 electrodes are placed on the skin of the patient P. An alternating current (feed current) with a predefined current intensity or with a current intensity known by measurement is fed between two electrodes of this series. These two electrodes are also called a stimulating electrode pair. The voltage, which results from the feed, is measured at the other electrodes. The impedance Z of the tissue between the two electrodes of the stimulating electrode pair is deduced according to Ohm's law as a quotient of the measured values for the voltage at the other electrodes and the known feed current intensity. The muscles and the blood in the body of the patient P can conduct the measuring current fed better than the pulmonary tissue can because muscles and blood contain more unbound ions. The two respective electrodes used as the stimulating electrode pair are changed in the course of time at a high frequency, and each electrode belongs to the stimulating electrode pair during a respective part of the measurement time period.

The higher the air content in a region of the lungs, the higher is the measured impedance in that region. The air content and hence the impedance increase in a region, as a rule, during the inhalation (generated by intrinsic breathing activity and/or by mechanical ventilation), and they decrease again during exhalation. The regional difference between the impedance at the end of the inhalation and the impedance at the end of the exhalation is correlated with the regional change in the air content in the lungs. A linear relationship, which is determined, e.g., in advance during a calibration, may be used as this correlation. The EIT measuring method therefore yields an image of the regional changes of the air content in the lungs in the body.

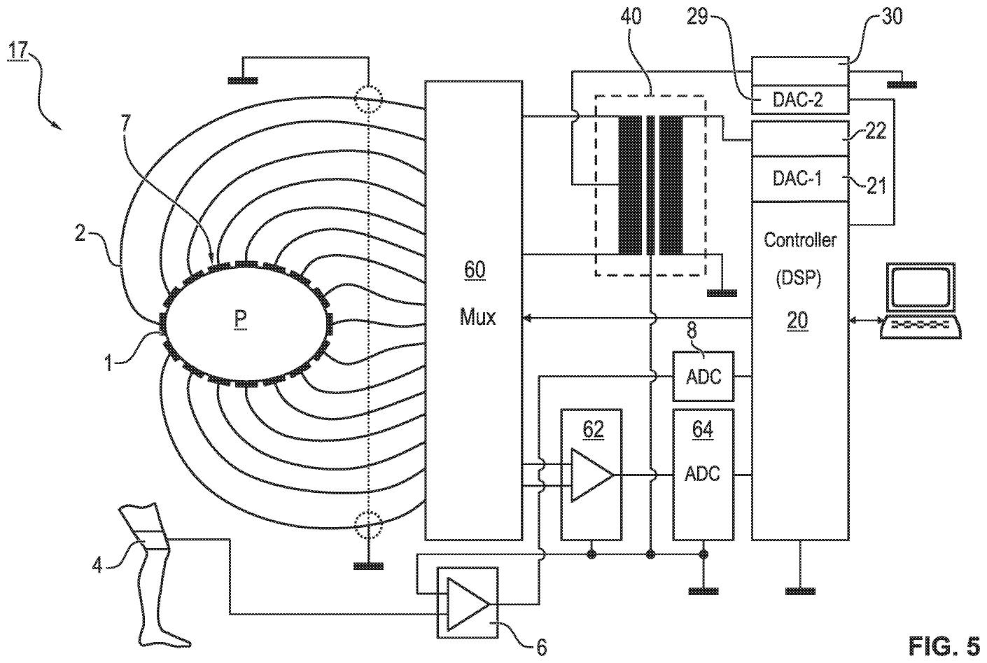

FIG. 5 shows as an example an EIT measuring device 17 as it is described, e.g., in DE 10 2005 031 752 B4 (corresponding U.S. Pat. No. 7,941,210 B2 is hereby incorporated by reference). EIT stands for electrical impedance tomography. An EIT belt 7, which belongs to the EIT measuring device 17, is placed around the body of the patient P. This EIT belt 7 comprises a plurality of measuring electrodes 1, specifically 16 measuring electrodes 1 in the example being shown.

Each measuring electrode 1 is connected via a respective cable 2 to a switch or multiplexer 60. The switch or multiplexer 60 applies an a.c. signal to two respective measuring electrodes 1, which will then act as a stimulating electrode pair. The selection of the stimulating electrode pair rotates around the body of the patient P. The remaining 14 measuring electrodes 1 act as measuring electrodes. The voltage signals of the measuring electrodes 1 are fed via the switch or multiplexer 60, via a difference amplifier 62 and via an analog-digital converter 64 to a control and analysis unit 20. The control and analysis unit 20 generates from the voltage signals an image of the regional distribution of the air content in the lungs. To generate this image, the control and analysis unit 20 uses a suitable method for image reconstruction. The generated image can be called an electrical impedance tomography image (EIT image).

The two respective activated measuring electrodes 1 are supplied with alternating current by an a.c. power source 22. The control and analysis unit 20 is connected via a digital-analog converter 21 to the a.c. power source 22 and it actuates same. The alternating current of the a.c. power source 22 is separated galvanically from the switch or multiplexer 60 by means of an isolation transformer or transformer 40.

For example, a measuring electrode 4, which measures the common mode signal on the body of the patient P, is attached to the right leg of the patient P. The signals of the measuring electrode 4 are fed to the control and analysis unit 20 via a measuring amplifier 6 and an analog-digital converter 8 to the control and analysis unit 20. The control and analysis unit 20 comprises one or more processors and memory and is connected to a compensation a.c. power source 30 via a digital-analog converter 29. The control and analysis unit 20 actuates the compensation a.c. power source 30 as to phase and amplitude such that the symmetry of the primary a.c. power source 22 is detuned on the secondary side of the isolation transformer 40, the detuning being such that the common mode signal on the body of the patient P is minimized. The control and analysis unit 20 uses for this regulation the common mode signal, which is measured by the measuring electrode 4 and is subsequently amplified.

The regional change in the impedance and hence the regional change in the air reserve in the lungs of the patient P are determined by means of such an EIT measuring device 17. The regional compliance C_{Lung}[Reg] is sought, and the regional compliance is related to a region Reg of the lungs of the patient P. The calculation of the respective compliance shall be carried out for a plurality of regions or even for each region of the lungs. The region Reg or each region Reg is selected to be such that the compliance in this region at a given time can be considered to be equal for the entire region. The regional compliance of the respiratory system of the patient P is designated by Cᵣₑₛₚ[Reg]. The regional compliance of a lung region may vary over time. In one embodiment, each pixel of the EIT image generated for the lungs is a respective region Reg. Larger regions, i.e., regions with a plurality of pixels of the EIT image, are possible as well. As was already described above, the compliance depends on the change in pressure and the brought-about volume change. The brought-about change in pressure is thus determined, as was just described, by the EIT measuring device 17.

The following lung mechanical model equations are used in the exemplary embodiment:

Cᵣₑₛₚ[Reg]=ΔZ[Reg]/ΔP_{aw},

C_{Lung}[Reg]=ΔZ[Reg]/ΔPₜₚ,

C_{ew}[Reg]=ΔZ[Reg]/ΔPₑₛ.

Here, ΔZ[Reg] designates the change in the electrical resistance (impedance) in the region Reg, i.e., the change relative to a predefined reference value. This regional change in the impedance is determined, as was just described, by the EIT measuring device.

To determine the regional compliance C_{Lung}[Reg] of the region Reg of the lungs, measurements are carried out at different settings of the ventilator 9. In one embodiment, the ventilator 9 carries out a maneuver automatically. It is possible that a command variable for this maneuver, which variable is variable over time, is predefined by a person, who is monitoring the maneuver. The end-expiratory pressure PEEP is set in this maneuver at first at a relatively high value, e.g., at the highest possible value, at which the respiratory system of the patient P will not be damaged with certainty. As a result, the respiratory system of the patient P is opened. The end-expiratory pressure PEEP is then set step by step, i.e., incrementally, at a value that is lower than the value set before. Even at the minimal set value the risk is low that the lungs collapse. A determination process is carried out at each set value for the end-expiratory pressure PEEP. For example, the end-expiratory pressure PEEP is set one after another at the eleven values 25 cm H₂O, 23 cm H₂O, . . . , 5 cm H₂O.

It is, of course, possible to reverse the procedure and to start at first with a lower value and then to increase the value step by step. However, the respiratory system of the patient P is not opened at the beginning of the maneuver in this alternative.

Each determination process at a set value for the end-expiratory pressure PEEP comprises the following steps, which are carried out by the control device 16 automatically:
The airway pressure P_{aw} as well as the pressure Pₑₛ in the esophagus Sp are measured repeatedly. The scanning frequency is preferably so high that the two pressures P_{aw} as well as Pₑₛ are measured at least once during the time period between the end of an inhalation process and the beginning of the next exhalation process as well as during the time period between the end of an exhalation process and the beginning of the next inhalation process. A measurement is, as a rule, more reliable during these two time periods than during another time period, because only a relatively small quantity of air and ideally no air whatsoever flows into the lungs or out of the lungs of the patient P during these two time periods, so that the two pressures remain approximately constant during these time periods.
The transpulmonary pressure is deduced in one embodiment according to the calculation rule Pₜₚ=P_{aw}−Pₑₛ, i.e., with the use of measured values of the sensors 3 and 10. According to this calculation rule, values for the end-inspiratory transpulmonary pressure EIPₜₚ and for the end-expiratory transpulmonary pressure EEPₜₚ are deduced.
The difference ΔPₜₚ=EIPₜₚ−EEPₜₚ is deduced as the value indicative of the pressure that brings about this change in the volume of the lungs. Another form of this deduction is the following calculation rule: ΔPₜₚ=(EIP+EIPₑₛ)−(PEEP−EEPₑₛ).
In another embodiment, the difference ΔPₜₚ is calculated according to the calculation rule ΔPₜₚ=(eelv2−eelv1)/(peep2−peep1). Here, peep1 is the current value and peep2 is the previous value for PEEP.
In addition, the regional change ΔZ[Reg] of the electrical resistance is deduced, for which measured values of the EIT measuring device 17 are used.
The regional compliance C_{Lung}[Reg] of the lungs in a region Reg is deduced according to the calculation rule C_{Lung}[Reg]=ΔZ[Reg]/ΔPₜₚ.

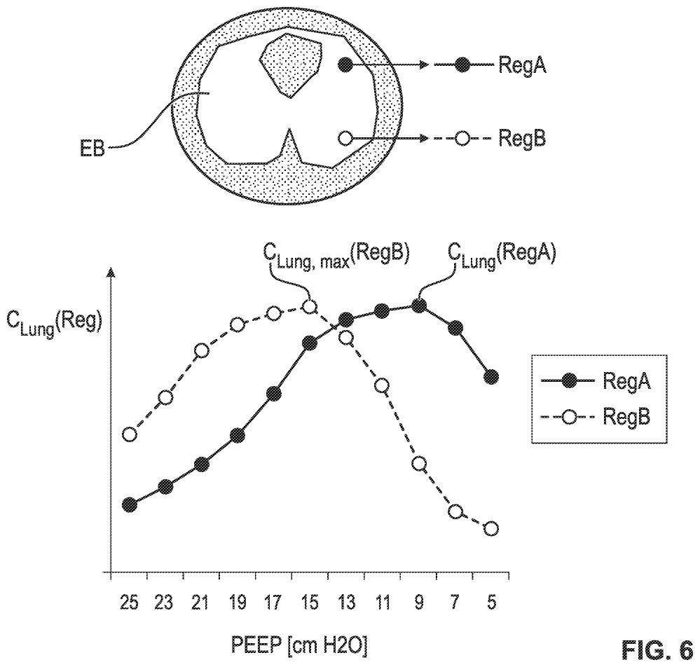

The top part of FIG. 6 illustrates two exemplary regions RegA and RegB of the lungs of the patient P. The patient P is usually lying in the dorsal position during a mechanical ventilation, and the intrinsic weight of the patient P acts on the body of the patient P thanks to the force of gravity, and the body extends at right angles to the force of gravity. The region RegA is located closer to the heart, and the region RegB is closer to the back of the patient P. With the patient in a lying position, the intrinsic weight of the body acts markedly more strongly on the region RegB than on the region RegA. These two exemplary regions RegA, RegB can be seen in the EIT image EB of the lungs.

The bottom part of FIG. 6 shows a diagram. The respective set value of the end-expiratory pressure PEEP in cm H₂O is shown on the x-axis and the regional compliance C_{Lung}[Reg], which was deduced just as described, is shown on the y-axis. Two curves C_{Lung}[RegA] and C_{Lung}[RegB] are shown.

It can be seen that the regional compliance C_{Lung}[RegA], C_{Lung}[RegB] assumes a maximum C_{Lung,max}[RegA], C_{Lung,max}[RegB] as a function of the set end-expiratory pressure PEEP.

This maximum varies, as a rule, from one region of the lungs to the next. An essential reason for this is that the effects of the intrinsic weight of the body of the patient P on these regions are different. Other influencing factors may be the regional inflammation, inactive surfactant or edemas. The value of the end-expiratory pressure PEEP at which the regional compliance C_{Lung}[Reg] assumes the maximum for this region will be designated by peepₘₐₓ[Reg] below. The maximum varies, as a rule, from one region to the next.

In one embodiment a respective value for the relative stiffness S_{Lung,rel}[Reg] is calculated relative to the maximum, for example, according to the formula

S_{Lung,rel}[Reg](peep)={C_{Lung,max}[Reg]−C_{Lung,max}[Reg](peep)}/C_{Lung,max}[Reg]*100%={1−E_{Lung,max}[Reg]/E_{Lung,max}[Reg](peep)}*100%

for reach region Reg of the lungs and for each set value peep of the end-expiratory pressure PEEP.

C_{Lung,max}[Reg](peep) is the regional compliance and E_{Lung,max}[Reg](peep) is the regional elastance at the peep value for PEEP.

The lower the regional compliance, the higher is this relative stiffness.

In one embodiment of mechanical ventilation, collapse of the lungs of the patient P shall primarily be prevented. A regional collapse value K_{Lung,rel}[Reg] is calculated in this application according to the calculation rule
K_{Lung,rel}[Reg](peep)=S_{Lung,rel}[Reg](peep) if peep>=peepₘₐₓ[Reg] and
K_{Lung,rel}[Reg](peep)=0 if peep<peepₘₐₓ[Reg].
   A cumulative collapse value K_{Lung,rel}(peep) is calculated according to the calculation rule
K_{Lung,rel}(Peep)=Σ{K_{Lung,rel}[Reg](peep)*C_{Lung,max}[Reg]}/ΣC_{Lung,max}[Reg].
   Summation is carried out in this case over all regions Reg of the lungs. This cumulated collapse value depends on the value of the end-expiratory pressure PEEP. The ventilator 9 is set at the value peep for PEEP that leads to the highest cumulated collapse value K_{Lung,rel}(peep).

Overdistention of the lungs shall be primarily prevented in another embodiment. A regional hyperdistention value is calculated in this other application according to the calculation rule
Ü_{Lung,rel}[Reg](peep)=S_{Lung,rel}[Reg](peep) if peep<peepₘₐₓ[Reg] and
Ü_{Lung,rel}[Reg](peep)=0 if peep>=peepₘₐₓ[Reg].
   A cumulative hyperdistention value Ü_{Lung,rel}(peep) is calculated according to the calculation rule
Ü_{Lung,rel}(peep)=Σ{Ü_{Lung,rel}[Reg](peep)*C_{Lung,max}[Reg]}/ΣC_{Lung,max}[Reg],
   in which summation is carried out again over all lung regions taken into consideration. Likewise, the ventilator 9 is set at the value that leads to the highest cumulated hyperdistention value Ü_{Lung,rel}(peep).

These two embodiments may be combined with one another.

While specific embodiments of the invention have been shown and described in detail to illustrate the application of the principles of the invention, it will be understood that the invention may be embodied otherwise without departing from such principles.

1 16 measuring electrodes of the EIT belt 7

2 Cables, which connect the measuring electrodes 1 to the multiplexer 60

3 Pneumatic sensor in front of the mouth of the patient P; it measures the airway pressure P_{aw} and optionally the volume rate of flow Vol′; it acts as an airway pressure sensor

3.1 Transducer of the sensor 3; it branches off air from the ventilation circuit

3.2 Pressure sensor proper of the sensor 3; it receives fluid streams from the transducer 3.1

4 Measuring electrode at the leg of the patient P; it measures a common mode signal of the body of the patient P against ground

5.1.1, 5.1.2 Pericardial pair of measuring electrodes on the skin of the patient P

5.2.1, 5.2.2 Pair of measuring electrodes on the skin of the patient P near the diaphragm

6 Measuring amplifier

7 EIT belt, placed around the body of the patient P; it comprises a plurality of (e.g., 16) measuring electrodes 1 and a respective cable 2 each per measuring electrode 1; it belongs to the EIT measuring device 17

8 Analog-digital converter

9 Ventilator; it ventilates the patient P mechanically; comprises the display unit 12

10 Probe in the esophagus Sp of the patient P; it measures the esophageal pressure Pₑₛ and optionally the gastric pressure P_{ga}

11 Connection piece in the mouth of the patient P, connected to the measuring catheter 14 in the esophagus Sp

12 Display unit of the ventilator 9; it comprises a display screen

13 Gastric probe in the stomach Ma of the patient P; it measures the gastric pressure P_{ga}

14 Measuring catheter in the esophagus Sp of the patient P; connected to the connection piece 11

15 Sensor at the ventilator 9; it measures the volume rate of flow Vol′

16 Control device; it receives signals from the EIT measuring device 17 and from the sensors 3, 10, 15

17 EIT measuring device; it comprises the EIT belt 7—EIT means electrical impedance tomography

20 Control and analysis unit of the EIT measuring device 17

21 Digital-analog converter

29 Digital-analog converter

30 Compensation a.c. power source

60 Multiplexer, to which the cables are connected

C_{ew} Average compliance of the chest wall of the patient P

C_{Lung} Average compliance of the lungs of the patient P

C_{Lung}[Reg] Regional compliance of the lungs in region Reg

C_{Lung}[Reg](peep) Value for the peep value of the regional compliance of the lungs in the region Reg

C_{Lung,max}[Reg] Maximum regional compliance of the lungs in region Reg

Cᵣₑₛₚ Average compliance of the respiratory system comprising the lungs and the chest wall of the patient P

Cᵣₑₛₚ[Reg] Regional compliance of the respiratory system in a region Reg

ΔP Change in the pressure present at the lungs
ΔP_{aw} Difference between the end-inspiratory pressure EIP and the end-expiratory pressure PEEP
ΔPₑₛ Difference between the end-inspiratory pressure EIPₑₛ and the end-expiratory pressure EEPₑₛ
ΔPₜₚ Difference between the end-inspiratory transpulmonary pressure EIPₜₚ and the end-expiratory transpulmonary pressure EEPₜₚ
ΔVol Change in the lung volume, which is brought about by the change ΔP in the pressure present at the lungs
ΔZ[Reg] Change in the electrical resistance (impedance) in the lung region Reg
EEPₑₛ End-expiratory pressure in the esophagus Sp, i.e., pressure Pₑₛ at the end of an exhalation process
EIP End-inspiratory pressure at the airway of the patient P, i.e., pressure P_{aw} at the end of an inhalation process
EIPₑₛ End-inspiratory pressure in the esophagus Sp, i.e., pressure Pₑₛ at the end of an inhalation process
EB EIT image of the lungs of the patient P
EEPₜₚ End-expiratory transpulmonary pressure, i.e., pressure Pₜₚ at the end of an exhalation process
EIPₜₚ End-inspiratory transpulmonary process, i.e., transpulmonary pressure Pₜₚ at the end of an inhalation process
K_{Lung,rel}[Reg] Regional collapse value in region Reg
Lu Lungs of the patient P
Ma Stomach of the patient P
P Patient, who is ventilated mechanically and breathes spontaneously, has the lungs Lu, the stomach Ma, the esophagus Sp and the diaphragm Zw
P_{aw} Pneumatic pressure at the airway (airway pressure); it is measured by means of the sensor 3
P_{di} Pressure at the diaphragm, determined as the difference P_{ga}−EEPₑₛ
Pₑₛ Pneumatic pressure in the esophagus Sp of the patient, determined by means of the probe 10
P_{ga} Gastric pressure in the stomach Ma of the patient P; it is measured by means of the probe 10 or of a gastric probe
Pₜₚ Transpulmonary pressure, preferably determined as the difference P_{aw}-Pₑₛ
PEEP End-expiratory pressure at the airway, i.e., pressure P_{aw} at the end of an exhalation process; it can be set at the ventilator 9
peep Value for PEEP
peepₘₐₓ[Reg] The value of PEEP at which the regional compliance C_{Lung}[Reg] for region Reg assumes the maximum
RegA, RegB Exemplary regions of the lungs of the patient P
S_{Lung,rel}[Reg](peep) Relative stiffness of the region Reg of the lungs at the value peep for PEEP
Sp Esophagus of the patient P; it accommodates the probe 10
Tb Tidal image of the lungs of the patient P, generated by means of the EIT measuring device 17
Ü_{Lung,rel}[Reg] Regional hyperdistention value in region Reg
Vol′ Volume rate of flow
Zw Diaphragm of the patient P

What is claimed is:

1. A device for determining for each lung region of a plurality of different given lung regions of the lungs of a patient a respective value indicative of a regional lung compliance of the lung region, the device comprising:
an EIT measuring device configured to measure for each lung region of the plurality of different lung regions a value indicative of a respective change in volume of the lung region by applying electrical impedance tomography;
a pneumatic airway pressure sensor configured to measure a value indicative of a pressure, which is variable over time, at an airway of the patient; and
a data processing control device configured:
to determine for each lung region of the plurality of different lung regions a respective value indicative of a difference between an end-inspiratory volume and an end-expiratory volume for each of the lung region using signals of the EIT measuring device;
to determine a value indicative of a difference between an end-inspiratory transpulmonary pressure present at the lungs and an end-expiratory transpulmonary pressure present at the lungs using signals of the airway pressure sensor; and
to calculate for each lung region of the plurality of different lung regions a quotient of the volume difference of the lung region and the pressure difference present at the lungs as the value indicative of a regional lung compliance of the lung region.

2. A device in accordance with claim 1, further comprising a pneumatic esophageal pressure sensor configured to measure a value indicative of an esophageal pressure, which is variable over time, in the esophagus of the patient, wherein the control device is configured:
to determine the end-inspiratory transpulmonary pressure as a difference between the end-inspiratory airway pressure and the end-inspiratory esophageal pressure; and
to determine the end-expiratory transpulmonary pressure as a difference between the end-expiratory airway pressure and the end-expiratory esophageal pressure.

3. A device in accordance with claim 1, wherein:
the device is configured to be in a data connection with a ventilator, which is configured to mechanically ventilate the patient;
the ventilator is configured to mechanically ventilate the patient such that an end-expiratory pressure at the airway of the patient assumes a predefined value;
the control device is configured to actuate the ventilator such that the actuated ventilator carries out a mechanical ventilation such that the end-expiratory pressure first assumes a predefined first value and subsequently assumes at least one predefined second value, which is different from the first value.

4. A device in accordance with claim 3, wherein the control device is configured to determine for each lung region a respective end-expiratory pressure value indicative of the regional lung compliance resulting from the first and second values of the end-expiratory pressure.

5. A device in accordance with claim 3, wherein:
the control device is configured to calculate a required operating parameter of the end-expiratory pressure at the airway;
the control device is configured to calculate the required operating parameter depending on a value indicative of the overall lung compliance; and
the control device is configured to cumulate over the regional compliance values of the lung regions for calcuating the overall lung complicance.

6. A device in accordance with claim 3, wherein:
the device is configured to determine a value indicative of the respective end-expiratory lung volume at the first value and at the second value of the end-expiratory pressure; and
the device is configured to calculate, as the value indicative of the difference between the end-inspiratory transpulmonary pressure and the end-expiratory transpulmonary pressure, the quotient of the difference between the two measured end-expiratory lung volumes and the difference between the two set values for the end-expiratory pressure.

7. A mechanical ventilation system comprising:
a device comprising:
an EIT measuring device configured to measure for each lung region of a plurality of different given lung regions of the lungs of a patient a value indicative of a respective change in volume of the lung region by applying electrical impedance tomography;
a pneumatic airway pressure sensor configured to measure a value indicative of a pressure, which is variable over time, at an airway of the patient; and
a data processing control device configured:
to determine for each lung region of the plurality of different lung regions a respective value indicative of a difference between an end-inspiratory volume and an end-expiratory volume of the lung region using signals of the EIT measuring device;
to determine a value indicative of a difference between an end-inspiratory transpulmonary pressure present at the lungs and an end-expiratory transpulmonary pressure present at the lungs using signals of the airway pressure sensor; and
to calculate for each lung region of the plurality of different lung regions a quotient of the volume difference of the lung region and the pressure difference present at the lungs as the value indicative of a regional compliance of the lung region; and
a ventilator configured to carry out a mechanical ventilation of the patient as a function of determined regional compliance values of the lung regions of the plurality of different lung regions.

8. A mechanical ventilation system in accordance with claim 7, wherein the device further comprises a pneumatic esophageal pressure sensor configured to measure a value indicative of an esophageal pressure, which is variable over time, in the esophagus of the patient, wherein the control device is configured:
to determine the end-inspiratory transpulmonary pressure as a difference between the end-inspiratory airway pressure and the end-inspiratory esophageal pressure; and
to determine the end-expiratory transpulmonary pressure as a difference between the end-expiratory airway pressure and the end-expiratory esophageal pressure.

9. A mechanical ventilation system in accordance with claim 7, wherein the control device is configured to actuate the ventilator such that the actuated ventilator carries out a mechanical ventilation such that an end-expiratory pressure assumes at first a predefined first value and, subsequent to assuming the predefined first value, at least one predefined second value, which is different from the first value.

10. A mechanical ventilation system in accordance with claim 9, wherein the control device is configured to determine for each lung region a respective end-expiratory pressure value indicative of the regional lung compliance resulting from the first and second values of the end-expiratory pressure.

11. A mechanical ventilation system in accordance with claim 9, wherein:
the control device is configured to calculate a required operating parameter of the end-expiratory pressure at the airway;
the control device is configured to calculate the required operating parameter depending on a value indicative of the overall lung compliance; and
the control device is configured to cumulate over the regional compliance values of the lung regions for calcuating the overall lung complicance.

12. A mechanical ventilation system in accordance with claim 9, wherein:
the device is configured to determine a value indicative of the respective end-expiratory lung volume at the first value and at the second value of the end-expiratory pressure; and
the device is configured to calculate, as the value indicative of the difference between the end-inspiratory transpulmonary pressure and the end-expiratory transpulmonary pressure, the quotient of the difference between the two measured end-expiratory lung volumes and the difference between the two set values for the end-expiratory pressure.

13. A process for determining for each lung region of a plurality of different lung regions of a patient a respective value indicative of a regional compliance of the lung region, wherein the process is carried out with a device, which comprises an EIT measuring device configured to measure a value indicative of a change in volume of a lung region; a pneumatic airway pressure sensor configured to measure a value indicative of a pressure, which is variable over time, at the airway of the patient, the process comprising the steps of:
determining a value indicative of the difference between an end-inspiratory transpulmonary pressure present at the lungs and an end-expiratory transpulmonary pressure present at the lungs;
measuring, with the EIT measuring device for each lung region of the plurality of different lung regions, a change in volume of the lung region by electrical impedance tomography;
for each lung region of the plurality of different lung regions determining a value indicative of a difference between an end-inspiratory volume and an end-expiratory volume of the lung region using signals of the EIT measuring device; and
for each lung region of the plurality of different lung regions calculating a quotient of the volume difference for the lung region and the pressure difference present at the lungs as the value indicative of the regional compliance of the lung region.

14. A process according to claim 13, wherein:
the device comprises a data processing control device configured to execute a computer program;
the control device is arranged to receive signals from the EIT measuring device for measuring a value indicative of the change in the volume of the lung region;
the control device is arranged to receive signals from the pneumatic airway sensor for measuring the value indicative of the pressure; and
the control device is arranged to perform the process steps when receiving signals from the EIT measuring device and the airway sensor.

15. A process according to claim 13, wherein the control device is activated by a signal sequence that causes the control device to carry out at least some of the process steps.
